(19) United States
(12) Patent Application Publication
(10) Pub. No.: US 2003/0013975 A1
(43) Pub. Date: Jan. 16, 2003

(54) Method of selling a continuous mode blood pressure monitor

(76) Inventor: Massi E. Kiani, Laguna Niguel, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 09/904,885

(22) Filed: Jul. 12, 2001

(51) Int. Cl. A61B 5/02

(52) U.S. Cl. 600/485; 600/490; 600/500

(57) ABSTRACT

A blood pressure monitor has a noninvasive continuous blood pressure measurement mode and a non-continuous blood pressure measurement mode. At the time of a first sale, the noninvasive continuous blood pressure measurement mode is disabled. Thus, the blood pressure monitor operates only in the non-continuous measurement mode. At a later time, a sensor is sold to generate a sensor signal for noninvasive continuous blood pressure measurements, thereby enabling the continuous measurement mode of the blood pressure monitor.

### BACKGROUND OF THE INVENTION

1. Field of the Invention

The present invention relates to a method for selling a blood pressure monitor that has a continuous mode and a non-continuous mode for measuring a patient's blood pressure.

2. Description of the Related Art

In the field of medicine, constant monitoring of a patient's blood pressure is needed because blood pressure is affected by the body's reaction during and after surgery. Continuous measurement of blood pressure has been linked to reduced risk of heart attack and stroke. As opposed to non-continuous measurement of blood pressure where measurements are taken at intervals, continuous measurements generally are beat-to-beat measurements and thereby provide more accurate and reliable indications of a patient's well being.

Two general methods have been developed for measuring blood pressure. The two methods are categorized as invasive and noninvasive. The invasive method places a catheter within the patient's body to receive continuous measurements. The invasive method has disadvantages such as the risk of embolization, infection, bleeding, and vessel wall damage.

Conventional, noninvasive methods avoid many of the risks posed by invasive methods. An exemplary conventional non-continuous noninvasive method is described in U.S. Pat. No. 4,677,984 where an occlusive cuff detects momentary increases in pressure caused by passage of blood through an artery beneath the cuff. But the conventional occlusive cuff is less accurate than the invasive method because blood pressure is only recorded at intermittent intervals in order to provide a sampling to process a waveform. U.S. Pat. No. 5,590,649 combines the advantages of the non-continuous noninvasive occlusive cuff of U.S. Pat. No. 4,677,984 and invasive continuous procedures without involving the risks of embolization, infection, bleeding, and vessel wall damage. U.S. Pat. No. 5,590,649 describes the use of a noninvasive sensor positioned over an artery and calibrated with use of an occlusive cuff. U.S. Pat. Nos. 4,677,984 and 5,590,649 are incorporated by reference herein in their entirety.

As discussed above, blood pressure monitors are available that employ noninvasive sensors to produce continuous measurements, thereby advantageously avoiding the problems associated with invasive procedures or non-continuous blood pressure measurements. However, hospitals and other medical service providers are reluctant to invest in blood pressure monitors with noninvasive sensors because the invasive arterial lines on conventional monitors provide data other than a patient's blood pressure. Hospitals are also skeptical about investing in new medical devices because many past advances in the medical devices industry have been short-lived. For example, hospitals have invested in new medical devices and later determined that the medical devices were not satisfactory. Because of the risk, hospitals are reluctant to buy new medical devices. In particular, hospitals have been reluctant to invest in blood pressure monitors with noninvasive sensors that provide continuous measurements.

### SUMMARY OF THE INVENTION

One aspect of the present invention is a method for selling devices that employ safer noninvasive blood pressure measurement methods to encourage hospitals and other medical facilities to use noninvasive blood pressure monitors. The method provides familiarity with the devices when used in a non-continuous mode. The method also provides a base for later implementation of the continuous mode for noninvasive blood pressure monitors as noninvasive monitors increase in acceptability. The method also encourages use of the non-continuous measurement mode while, for example, governmental approval is being sought for the continuous measurement mode. Further, the method provides time to research the potential for substituting a noninvasive blood pressure monitor to measure data ordinarily provided by an arterial line.

One aspect of the present invention is a method for using a continuous mode blood pressure monitor having a sensor input for receiving a sensor signal for continuous blood pressure measurement and a cuff for establishing a baseline blood pressure measurement to be used to calibrate continuous blood pressure measurements. At a first time, the blood pressure monitor is used without the sensor for providing a sensor signal so that the blood pressure monitor operates with the cuff to provide non-continuous measurements of blood pressure. At a second time, the blood pressure monitor is used with a sensor to provide the sensor signal to enable the continuous measurement mode.

Another aspect of the present invention is a method for selling a multi-mode blood pressure monitor that has a continuous measurement mode and a non-continuous measurement mode. At a first time, the multi-mode blood pressure monitor is sold with the continuous measurement mode disabled. At a later time, a sensor is sold to enable the continuous measurement mode of the multi-mode blood pressure monitor.

Another aspect of the present invention is a method for converting a non-continuous mode blood pressure monitor to a continuous mode blood pressure monitor by generating a sensor signal for continuous blood pressure measurements. The blood pressure monitor is manufactured with a non-continuous measurement mode and a continuous measurement mode, but the monitor initially has the continuous measurement mode disabled. A sensor is sold to enable the continuous measurement mode.

Another aspect of the present invention is a method for selling a sensor for generating a sensor signal for continuous blood pressure measurements. The sensor input is attachable to a continuous mode blood pressure monitor having a cuff for establishing a baseline blood pressure measurement to be used to calibrate continuous blood pressure measurements. The blood pressure monitor is initially sold with the continuous measurement mode disabled. At a time after the sale of the blood pressure monitor, the sensor is sold for generating the sensor signal to enable the continuous blood pressure measurements so that the blood pressure monitor is operable with the cuff to provide continuous measurements of blood pressure.

### BRIEF DESCRIPTION OF THE DRAWINGS

The foregoing aspects and other aspects of the present invention will be described in detail below in connection with the accompanying drawings in which:

FIG. 1 illustrates a noninvasive non-continuous measurement mode blood pressure monitor attached to a patient;

FIG. 2 illustrates a noninvasive continuous measurement mode blood pressure monitor attached to a patient;

FIG. 3 is a flowchart illustrating a method that a seller of blood pressure monitors can use to sell multi-mode blood pressure monitors; and

FIG. 4 is a flowchart illustrating a method that a medical service provider can use to operate multi-mode blood pressure monitors.

### DETAILED DESCRIPTION OF THE PREFERRED EMBODIMENT

As discussed above, hospitals and other medical service providers are reluctant to buy noninvasive continuous mode blood pressure monitors. In accordance with the present invention, the noninvasive blood pressure monitor is a multi-mode blood pressure monitor having a non-continuous blood pressure measurement mode and a continuous blood pressure measurement mode. In one embodiment, the seller of a multi-mode blood pressure monitor does not immediately sell monitors as multi-mode monitors. Rather, the seller continues to sell the monitor as a noninvasive non-continuous measurement mode blood pressure monitor that can be used in conjunction with invasive continuous measurement mode devices such as arterial lines. The medical service provider does not need to be concerned with the efficacy of the noninvasive non-continuous measurement mode working in conjunction with arterial lines because this device configuration has proven to be effective in the past. According to one embodiment, the multi-mode monitor has the noninvasive continuous measurement mode disabled at the time of the initial sales transaction. According to another embodiment, the monitor produces non-continuous measurements until a continuous sensor is attached. The seller of the multi-mode monitor can establish a base for the use of the multi-mode monitor as a noninvasive continuous mode measurement blood pressure monitor.

After the initial sales, the seller of the multi-mode blood pressure monitors can work within the scientific community to establish the efficacy of the noninvasive continuous measurement mode of the blood pressure monitor. The seller can also work with selected medical service providers to show that the continuous measurement mode operates satisfactorily. As the market acceptance increases and as the monitor gains a reputation for providing accurate noninvasive continuous measurements of blood pressure, other medical service providers, who initially were reluctant to invest in breakthrough medical devices because of lowered expectations from past performance, can be persuaded to purchase the ability to enable the noninvasive continuous blood pressure measurement mode of the monitor. For example, after the medical service providers are convinced about the efficacy of noninvasive continuous mode blood pressure monitors, the seller can sell a component to enable the continuous measurement mode of the blood pressure monitor. In the preferred embodiment described herein, the component advantageously includes a sensor that is attachable to the blood pressure monitor. The component can also include an integrated circuit or other electrical component that enables the continuous measurement mode. The component can be sold at a price that is relatively low in comparison to the blood pressure monitor in order to further encourage the service provider to enable the continuous measurement mode of the blood pressure monitors.

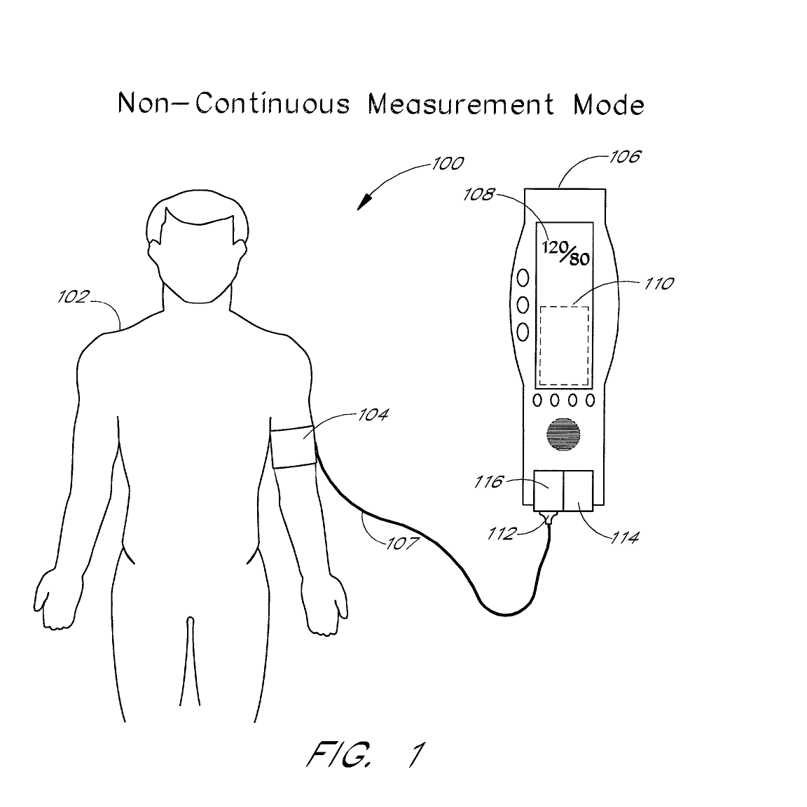

FIG. 1 illustrates a noninvasive non-continuous measurement mode configuration 100 as presently used by medical service providers, such as, for example, hospitals, to periodically measure blood pressure. The configuration 100 may be used in conjunction with invasive measurement devices (not shown) that provide continuous measurements. The noninvasive non-continuous measurement mode configuration 100 includes a cuff 104, a monitor 106, and a cable 107. According to one embodiment and as will be readily understood by a skilled artisan, the cuff 104 generally includes an inflatable bladder (not shown) which is filled to a pressure that temporarily occludes blood flow through an artery. Thereafter, one of several cuff methodologies may advantageously be employed in order to obtain blood pressure data while the cuff 104 deflates. For example, the auscultatory methodology employs sound sensing devices to detect the Korotkoff sounds of blood flow during cuff deflation, while the oscillometric methodology measures oscillations resulting from the direct coupling of the inflatable cuff 104 with the artery as the artery pulses.

Although disclosed with reference to auscultatory and ocillometric methodologies, the invention is not intended to be limited to a particular cuff device or cuff methodology. Rather, a skilled artisan will recognize from the disclosure herein a wide number of alternative cuff devices employing a wide number of cuff methodologies for determining blood pressure, such as, for example, the palpatory methodology focusing on when the finger pulse is first detected after cuff occlusion, the infrasound methodology attempting to detect low frequency vibrations after cuff occlusion, the ultrasound methodology tracking Doppler shifts, or the like. However, to facilitate a complete understanding of the invention, the remainder of the detailed description describes the invention as including the cuff 104 comprising an inflatable cuff 104 employing a version of the auscultatory methodology to determine non-continuous blood pressure measurements.

Therefore, to facilitate the auscultatory methodology, the inflatable cuff 104 also generally includes a transducer (not shown). Moreover, as shown in FIG. 1, the inflatable cuff 104 is attached to a patient 102 with the inflatable cuff 104 encircling a portion of the patient's body (e.g., the upper arm, as shown in FIG. 1). Generally, the transducer is positioned between the bladder and the patient's body. The inflatable cuff 104 is attached to the monitor 106 via the cable 107. The cable 107 advantageously has a pneumatic portion for providing air pressure from the monitor 106 to the bladder in the inflatable cuff 104 and has an electrically conductive portion for communicating electrical signals from the transducer to the monitor 106. The cable 107 has a connector 112 at the opposite end from the inflatable cuff 104. The connector 112 is removably engagable with an input connector 116 on the monitor 106 to provide both the pneumatic connection and the electrical connection between the cable 107 and the monitor 106.

The monitor 106 controls the air pressure applied to the inflatable cuff 104 to selectively inflate and deflate the bladder in the inflatable cuff 104. The monitor 106 also monitors the pressure sensed by the transducer in the inflatable cuff 104 to make periodic measurements of the blood pressure by determining the systolic and diastolic blood pressure in a manner known in the art. In particular, the monitor 106 pressurizes the inflatable cuff 104 to inflate the bladder around the patient's 102 arm to occlude blood flow. As the pressure from the inflatable cuff 104 is slowly reduced, the transducer within the inflatable cuff 104 senses when blood flow begins. This blood pressure measurement is recorded as the systolic pressure. After blood pressure is further reduced, the transducer in the inflatable cuff 104 senses when blood flow has been fully restored. This blood pressure measurement is recorded as the diastolic pressure. The inflating and deflating of the bladder in the inflatable cuff 104 and the recording of the systolic and diastolic pressures is defined as a measurement cycle. The measurement cycles can be repeated as needed (e.g., once every few minutes, once every few hours, or the like) to provide periodic measurements of the patient's blood pressure; however, it should be understood that the pressure applied to the patient's arm causes discomfort and also causes the blood flow to be occluded. Such occlusive measurements of blood flow generally are spaced apart by long intervals to minimize the discomfort and the occlusion of blood flow. Thus, occlusive measurement techniques are not suitable for providing continuous measurements of blood pressure.

The monitor 106 further includes a sensor input 114. The sensor input 114 is included to receive a sensor signal to enable the continuous measurement mode of the blood pressure monitor. However, in the configuration of FIG. 1, the continuous measurement mode is not enabled because the sensor input 114 is not connected.

The monitor 106 includes a digital display 108 that displays the results of the periodic measurements made using the inflatable cuff 104 in the manner discussed above. At the end of each measurement cycle, the digital display 108 changes to reflect the present systolic and diastolic blood pressure measurements. Alternatively, an additional screen may be provided to display a history of the past blood pressure measurements at the non-continuous measurement intervals.

The monitor 106 further includes a display or display portion 110 that is used to show the results of continuous blood pressure measurements when the continuous measurement mode is enabled, such as, for example, a blood pressure waveform or the like. In FIG. 1, the display 110 is shown as a blank screen to illustrate that the continuous measurement mode is not enabled. In an alternative embodiment (not shown), the monitor can also include an invasive continuous measurement mode, and the display 110 can be configured to display the results of the invasive measurement procedures. For example, in such an alternative embodiment, the monitor 106 can operate the inflatable cuff 104 in the manner described above to generate a baseline measurement of the blood pressure to be used to calibrate an arterial line (not shown) that can be connected between the patient 102 and the monitor 106. The arterial line can provide a beat-to-beat blood pressure measurement to be sent to the monitor 106, which displays an analog beat-to-beat measurement of blood pressure as opposed to the periodic blood pressure measurement displayed by the digital display 108. Thus, the monitor 106 can be used effectively with conventional invasive continuous measurement procedures with the noninvasive continuous measurement mode disabled while the noninvasive continuous measurement mode is being tested for approval.

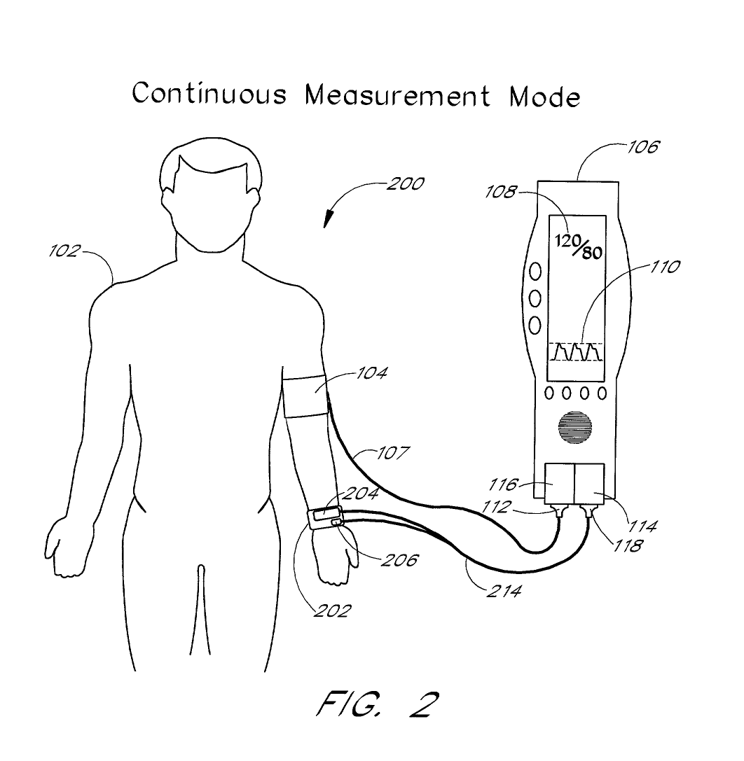

FIG. 2 illustrates a noninvasive continuous measurement mode blood pressure monitor configuration 200 that provides continuous measurements of blood pressure without invasive procedures. The noninvasive continuous measurement mode blood pressure monitor configuration 200 includes the inflatable cuff 104, the monitor 106 and the cable 107, as described above. In addition, the configuration 200 includes a second cable 214 and a sensor 202. The sensor 202 is attached to one end of the second cable 214. The second cable 214 has a connector 218 attached to the opposite end. The connector 218 engages the sensor input 114 of the monitor 106 to provide electrical communication from the sensor 202 to the monitor 106.

The inflatable cuff 104 is attached to the patient 102 and operates in conjunction with the monitor 106 in a similar fashion as discussed in FIG. 1. After the sensor 202 is attached to the patient 102 and is connected to the monitor 106, the inflatable cuff 104 is operated as described above to provide a baseline measurement of the patient's blood pressure. The baseline measurement is used to calibrate the noninvasive continuous mode measurements made using the sensor 202.

The sensor 202 can be attached to the monitor 106 at any time after the blood pressure monitor 106 is initially put into service to operate in the non-continuous measurement mode described above in connection with FIG. 1. Alternatively, after the blood pressure monitor 106 has been approved and accepted for use by the medical service providers, the sensor 202 can be sold with or attached to the blood pressure monitor 106 for the initial use of the blood pressure monitor 106.

In the illustrated embodiment, the noninvasive sensor 202 includes an exciter 204 and a transducer 206. The transducer 206 is a distinct transducer from the transducer (not shown) in the inflatable cuff 104. The exciter 204 induces a perturbation in the patient's 102 blood. The transducer 206 senses an effect of the perturbation which varies in response to changes in the patient's blood pressure. The transducer 206 can be any detector that senses an effect of the perturbation. In one embodiment, the transducer 206 senses hemoparameters other than blood pressure or other physiological parameters for continuous measurement or monitoring of a condition of the patient 102.

When the noninvasive continuous measurement mode is enabled, for example, when the sensor 202 is connected, the sensor 202 transmits the signals received by the transducer 206 to the sensor input 114 of the monitor 106 via the second cable 214 and the connector 118. The monitor 106 processes the signals from the sensor 202 to generate the results of the continuous measurements. The monitor 106 also displays the continuous measurements on the display 110. The processor 106 also advantageously displays the continuous measurements in a digital format in the digital display 108.

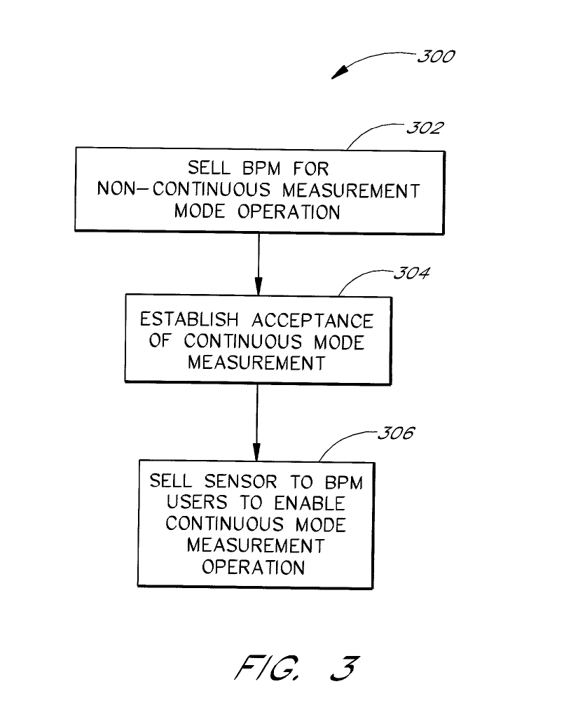

FIG. 3 illustrates a process 300 that can be used by a seller of blood pressure monitors to sell a noninvasive continuous mode measurement blood pressure monitor. As shown in a process block 302, the seller sells a blood pressure monitor 106 configured only for non-continuous blood pressure measurements, as illustrated by the configuration 100 in FIG. 1. A medical service provider (e.g., a doctor or a hospital) uses the blood pressure monitor 106 in the configuration 100 in the same manner that the medical service provider presently uses conventional blood pressure monitors. In particular, the bladder in the inflatable cuff 104 is inflated and deflated while the transducer monitors blood flow, as described above. The medical service provider can use the monitor 106 and the inflatable cuff 104 to provide baseline blood pressure measurements for invasive procedures such as arterial lines as it presently does in order to obtain continuous blood pressure measurements. Therefore, by purchasing the non-continuous measurement mode blood pressure configuration 100, the medical service provider conducts blood pressure measurements in a manner it knows to be reliable and can continue the practice of using arterial lines to collect data other than blood pressure measurements. By providing the blood pressure monitor 106 that gives the medical service provider the same functionality that the medical service provider already has, the seller of blood pressure monitors can persuade the medical service provider to purchase the blood pressure monitor 106 that can be modified later to have continuous blood pressure measurement capability.

The process 300 advances to a process block 304 where the seller of the blood pressure monitor establishes the acceptance of the noninvasive continuous blood pressure measurement mode of the blood pressure monitor 106 by extensive testing and by seeking any necessary approvals (e.g., FDA approvals) for using the blood pressure monitor 106 for continuous measurements. In the interim, the seller of the multi-mode blood pressure monitor 106 can conduct studies and provide the medical service provider with statistics regarding the advantages that the noninvasive continuous mode blood pressure monitor 106 has over invasive continuous mode blood pressure monitors. Further, as discussed above, the medical service provider uses the blood pressure monitor 106 to provide non-continuous measurements and thus becomes acquainted with the ease of use and the quality of the blood pressure monitor 106.

After acceptance of the noninvasive continuous blood pressure measurement mode is established, the process 300 advances to a process block 306 where the seller of the multi-mode blood pressure monitor 106 sells the sensor 202 to the medical service provider. The sensor 202 (or other enabling components) can be sold at a price which is relatively low in contrast to the blood pressure monitor 106. The sensor 202 is attached to the monitor 106, as described above, to enable the continuous measurement mode. Thus, unlike blood pressure monitors currently used by medical service providers, the original non-continuous measurement mode blood pressure monitor configuration 100 can be subsequently changed to the continuous measurement mode configuration 200 (FIG. 2) to enable the blood pressure monitor 106 to provide noninvasive continuous blood pressure measurements, through, for example, attaching the sensor 202.

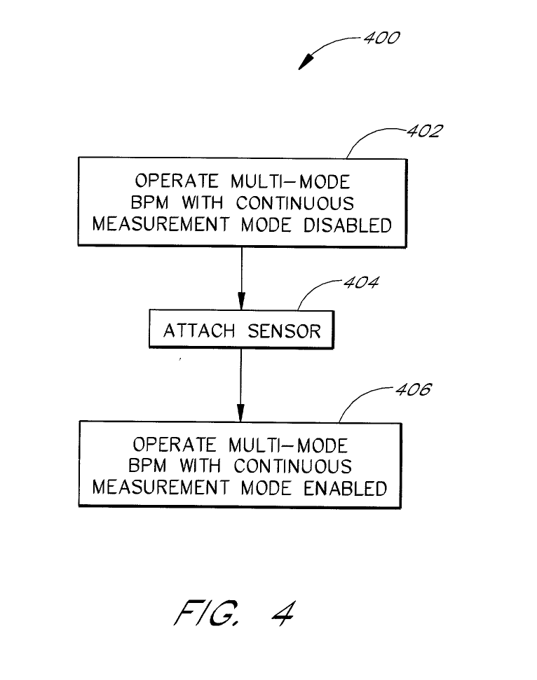

FIG. 4 illustrates a process 400 which can be used by a medical service provider to operate different modes of the blood pressure monitor 106. In a process block 402, the medical service provider operates the multi-mode blood pressure monitor configuration 100, as illustrated in FIG. 1. With the continuous measurement mode disabled, the medical service provider operates the blood pressure monitor in the same manner that the medical service provider presently uses conventional non-continuous blood pressure monitors. As discussed above with respect to FIG. 3, the medical service provider uses the blood pressure monitor 106 with the inflatable cuff 104 to obtain non-continuous blood pressure measurements. Further, the medical service provider can use the blood pressure monitor 106 in combination with invasive devices, such as arterial lines, in order to obtain continuous blood pressure measurements or other types of data. After a period of time, which may depend in part on the time required to convince the medical service provider of the efficacy of the noninvasive continuous measurement mode, the process 400 advances to a process block 404 where the medical service provider attaches the sensor 202 to the blood pressure monitor 106 to convert the configuration 100 of FIG. 1 to the configuration 200 of FIG. 2. The process 400 then advances to a process block 406 where the medical service provider operates the blood pressure monitor 106 in the continuous measurement mode illustrated in FIG. 2. By using the configuration 200 of FIG. 2, the medical service provider no longer needs to use invasive devices in order to make continuous blood pressure measurements. The medical service provider may still use invasive devices in order obtain other data. As discussed above, the blood pressure monitor 106 may be adapted to also work with invasive devices to obtain data other than blood pressure.

Although this invention has been described in terms of certain preferred embodiments, a skilled artisan will recognize other embodiments from the disclosure herein. For example, the cable 214 may advantageously comprise one or more independent electrical connections to the monitor 106 through one or more connectors 118 and one or more sensor inputs 114. Moreover, a skilled artisan will recognize from the disclosure herein that the inflatable cuff 104 may advantageously comprise a large number of commercially available non-continuous blood pressure sensing devices adapted to communicate a signal representative of the blood pressure of the patient 102.

Additionally, a skilled artisan will recognize from the disclosure herein that the monitor 106 may be adapted to receive multiple continuous signals from varying types of sensors, such as, for example, invasive sensors, non-invasive sensors, or both. Also, the sensor 202 may advantageously be adapted to produce an output signal consistent with that of conventional invasive technologies, thereby allowing the sensor 202 to be used with various types of monitors, or allowing the monitor to be adapted for use with invasive-type sensors.

Moreover, a skilled artisan will recognize from the disclosure herein that the monitor 106 may also be adapted to compute blood pressure measurements following varying types of measurement methodologies, such as, for example, pulsatile-velocity methodologies using, for example, an active perturbation of an artery, tonometric technologies, Finapress technologies from Ohmeda, or any additional technology known to the skilled artisan to produce measurements indicative of the blood pressure of a patient. According to one embodiment, the monitor 106 may be configurable to select a measurement methodology advantageously corresponding to or being compatible with the types of sensors connected to the monitor 106.

Other embodiments will also be apparent to those of ordinary skill in the art, including embodiments which do not provide all of the benefits and features set forth herein. Such embodiments are also within the scope of this invention. Accordingly, the scope of the present invention is defined only by reference to the appended claims.

What is claimed is:

1. A method of using a continuous multi-mode blood pressure monitor having a sensor input for receiving a sensor signal for continuous blood pressure measurement and a cuff for establishing a baseline blood pressure measurement to be used to calibrate continuous blood pressure measurements, the method comprising:
at a first time, using the blood pressure monitor without a sensor for providing a sensor signal so that the blood pressure monitor operates with the cuff to provide non-continuous measurements of blood pressure; and
at a second time, using a blood pressure monitor with a sensor for providing a sensor signal that enables the continuous measurement mode.

2. The method of claim 1, wherein pressure is applied to the cuff to occlude blood flow.

3. The method of claim 2, wherein a transducer determines when blood flow begins as pressure to the cuff is slowly reduced to provide non-continuous systolic measurements of blood pressure.

4. The method of claim 3, wherein the transducer detects when full blood flow is restored to provide non-continuous diastolic pressure measurements.

5. The method of claim 5, wherein the sensor includes an exciter, which induces a perturbation along the artery, and includes a transducer, which senses an effect of the perturbation that varies in response to changes in the patient's blood pressure.

6. The method of claim 5, wherein the exciter and the transducer are integrated into one unit.

7. The method of claim 6, wherein the exciter and the transducer are integrated into a wristband.

8. The method of claim 1, wherein the sensor is attached to the patient's forearm above the radial artery.

9. The method of claim 1, wherein the sensor is a noninvasive sensor.

10. The method of claim 1, further comprising at the second time:
exciting a perturbation in a patient's blood; and
sensing an effect of the perturbation that varies in response to changes in the patient's blood pressure.

11. The method of claim 1, wherein the cuff is a calibration device configured to provide a calibration signal representative of the patient's physiological hemoparameter.

12. The method of claim 1, wherein the cuff comprises an inflatable cuff.

13. The method of claim 1, wherein the cuff comprises an occlusive cuff.

14. A method of selling a multi-mode blood pressure monitor, comprising:
at a first time, selling a blood pressure monitor having a continuous measurement mode and a non-continuous measurement mode, the blood pressure monitor having the continuous measurement mode disabled; and
at a later time, selling a sensor to enable the continuous measurement mode.

15. The method of claim 14, wherein the sensor includes an exciter which induces a perturbation in a patient's blood, and a transducer which senses an effect of the perturbation that varies in response to changes in the patient's blood pressure.

16. The method of claim 14, wherein the exciter and the transducer are integrated into one unit.

17. The method of claim 14, wherein the exciter and the transducer are integrated into a wristband.

18. The method of claim 14, wherein the sensor is a noninvasive sensor.

19. The method as defined in claim 14, wherein said sensor is sold after the efficacy of the continuous measurement mode of the blood pressure monitor is established.

20. The method as defined in claim 19, wherein the sensor plugs into a connector sold with the blood pressure monitor at the first time.

21. A method of selling a continuous mode blood pressure monitor having a sensor input for receiving a sensor signal for continuous blood pressure measurements and having a cuff for establishing a baseline blood pressure measurement to be used to calibrate continuous blood pressure measurements, the method comprising:
at a first time, selling the blood pressure monitor without a sensor for providing the sensor signal so that the blood pressure monitor is operable only with the cuff to provide non-continuous measurements of blood pressure; and
at a second time, selling a sensor to provide the sensor signal to enable the continuous measurement mode.

22. A method of converting a non-continuous mode blood pressure monitor to a continuous mode blood pressure monitor by generating a sensor signal for continuous blood pressure measurements comprising:
providing a sensor to enable continuous measurement mode for a blood pressure monitor having a continuous measurement mode and a non-continuous measurement mode, said blood pressure monitor initially having the continuous measurement mode disabled.

23. A method of selling a sensor attachable to a multi-mode blood pressure monitor having a cuff for establishing a base line blood pressure measurement to be used to calibrate continuous blood pressure measurements, the method comprising:
at a time after the sale of the multi-mode blood pressure monitor with only a non-continuous measurement mode enabled, selling the sensor to generate a sensor signal for continuous blood pressure measurements so that the blood pressure monitor is operable with the cuff to provide continuous measurements of blood pressure.

24. A method of selling a blood pressure monitor having a non-continuous measurement mode and a continuous measurement mode comprising:
selling the blood pressure monitor with the continuous mode disabled so that the blood pressure monitor operably connected to a cuff can provide non-continuous measurements of blood pressure, and
at a time after the blood pressure monitor is sold, selling a sensor to attach to the blood pressure monitor to provide continuous measurements of blood pressure.

25. A method of using a multi-mode blood pressure monitor, comprising:
at a first time, using a blood pressure monitor having a continuous measurement mode and a non-continuous measurement mode, said blood pressure monitor having the continuous measurement mode disabled; and
at a later time, attaching a sensor to enable the continuous measurement mode.
